(19) United States
(12) Patent Application Publication
(10) Pub. No.: US 2019/0262605 A1
(43) Pub. Date: Aug. 29, 2019

(54) METHODS AND SYSTEMS OF INDUCING HYPERTHERMIA IN CANCER CELLS

(71) Applicant: WILLIAM MARSH RICE UNIVERSITY, Houston, TX (US)

(72) Inventors: Aydin BABAKHANI, Houston, TX (US); Hongming LYU, Houston, TX (US)

(73) Assignee: WILLIAM MARSH RICE UNIVERSITY, Houston, TX (US)

(21) Appl. No.: 16/333,392

(22) PCT Filed: Sep. 19, 2017
(86) PCT No.: PCT/US17/52163

§ 371 Date: Mar. 14, 2019

Related U.S. Application Data
(60) Provisional application No. 62/396,590, filed on Sep. 19, 2016.

(51) Int. Cl. A61N 1/36; A61B 18/14; A61B 5/053; A61B 18/08; A61B 5/00; A61N 1/372; A61N 1/375

(52) U.S. Cl. CPC A61N 1/36002; A61B 18/14; A61B 5/053; A61B 18/082; A61B 5/14539; A61N 1/37205; A61N 1/37211; A61N 1/37514; A61B 5/686

(57) ABSTRACT

Inducing hyperthermia in cancer cells. At least some of the example embodiments are methods including: charging a capacitor of a microchip device proximate to cells within the body, the charging by harvesting ambient energy by the microchip device; and when the energy on the capacitor reaches or exceeds a predetermined value inducing hyperthermia in the cells proximate to the microchip device using energy from the capacitor.

### CROSS-REFERENCE TO RELATED APPLICATIONS

This application claims priority to U.S. Patent Application Ser. No. 62/396,590 filed Sep. 19, 2016 titled “Pulsed Hyperthermia Based on Microchip Integrated Circuits.” The provisional application is hereby incorporated by reference in its entirety for all purposes.

### TECHNICAL FIELD

Various embodiments are directed to systems and methods of inducing hyperthermia in cells, such as cancer cells, within a body.

### BACKGROUND

There are several related art methods of inducing hyperthermia in cancer cells within a body. For example, in some cases radio frequency (RF) energy is sourced outside the body, directed into the body, and focused on the cancer cells. The RF energy causes localized heating and eventually cell death. However, RF energy is indiscriminate—the RF energy induces hyperthermia in all cells at which the RF energy is focused, and to some extent cells through which the RF energy propagates to reach the focal point. That is, RF energy induces hyperthermia in all cells, whether cancer cells or normal, healthy cells. In other cases, the tumor sites are very localized and it is difficult to perform the conventional RF hyperthermia.

### SUMMARY

The various embodiments are directed to systems and methods of inducing hyperthermia in cancer cells. More particularly, various embodiments are directed to wireless methods of determining the presence of cancer cells proximate to a microchip device, and inducing hyperthermia in the cells.

Example embodiments implantable medical device for inducing hyperthermia in cancer cells, the medical device comprising a substrate of semiconductor material, an energy harvesting circuit defined on the substrate, and an energy delivery circuit defined on the substrate. The energy harvesting circuit configured to extract electrical energy from energy propagating proximate the medical device and to store electrical energy in a capacitor). The energy delivery circuit electrically coupled to the energy harvesting circuit, and the energy delivery circuit configured to induce hyperthermia in cells proximate to the substrate.

Implementations of the invention can include one or more of the following features:

The energy delivery circuit further includes a resistive element defined on the substrate and electrically coupled to the energy harvesting circuit, and the energy delivery circuit is configured to apply heat to the tissue by conduction to the tissue of heat created from the resistive element.

The resistive element may be any of the following, or combinations thereof: a resistor; a transistor biased into an active region; a bipolar junction transistor biased into an active region; and a complementary metal-oxide semiconductor transistor biased into an active region.

The energy delivery circuit can further include a first set of electrodes on the substrate and electrically exposed, the first set of electrodes configured to selectively couple to the energy harvesting circuit. And the energy delivery circuit configured to induce hyperthermia by electrical current flow through the cancer cells by way of the first set of electrodes. In some cases the first set of electrodes are separated by 1000 microns or less, and in other cases the first set of electrodes are separated by 10 microns or less.

The communication circuit can further include a communication antenna defined on the substrate, the communication antenna operates at a frequency above 1 Mega Hertz (MHz). The communication circuit configured to receive the command from an external device by way of the communication antenna.

The energy harvesting circuit can further include an energy harvesting antenna defined on the substrate, a rectifier defined on the substrate and electrically coupled between the energy harvesting antenna and the capacitor, and a power management unit defined on the substrate and electrically coupled to the capacitor. The energy harvesting antenna has an operating frequency above 1 Mega Hertz (MHz). The configured to rectify alternating current energy from the energy harvesting antenna to create rectified energy stored in the capacitor. The power management unit configured to produce a regulated direct current (DC) voltage from rectified energy stored on the capacitor.

The energy harvesting can further include a set of conductive pads electrically exposed on the substrate, a rectifier defined on the substrate and electrically coupled between the second set of conductive pads and the capacitor, and a power management unit defined on the substrate and electrically coupled to the capacitor. The rectifier circuit configured to rectify alternating current energy flowing through the set of conductive pads to create rectified energy stored on the capacitor. The power management unit configured to produce a regulated DC voltage from the rectified energy stored on the capacitor.

The substrate of the implantable medical device in some cases defines a length greater than a width, and the width is 500 microns or less, and in particular cases 200 microns or less.

The implantable medical device can further include an encapsulant that fully encapsulates the substrate and devices defined on the substrate, the encapsulant electrically non-conductive.

The implantable medical device can further include a communication circuit defined on the substrate. The communication circuit electrically coupled to the energy harvesting circuit and the energy delivery circuit, and the communication circuit configured to receive a command originated external to the implantable medical device. The energy delivery circuit is configured to induce hyperthermia responsive to the command received by the communication circuit.

The implantable medical device can further include a sensing circuit defined on the substrate, the sensing circuit electrically coupled to the energy harvesting circuit and communicatively coupled to the communication circuit. The sensing circuit configured to sense a property of the cells proximate to or abutting the substrate. The property sensed by the sensing circuit can be any or all of: pH; resistivity; conductivity; impedance; transmittance; dielectric constant; and oxygen concentration or oxygen level.

Other example embodiments are methods of inducing hyperthermia in cancer cells within a body. The method may include charging a capacitor of a microchip device proximate to cells within the body (the charging by harvesting ambient energy by the microchip device), and when the energy on the capacitor reaches or exceeds a predetermined value inducing hyperthermia in the cells proximate to the microchip device using energy from the capacitor.

plementations of the method aspects of the invention can include one or more of the following features:

Charging the capacitor may further include harvesting electrical energy from electromagnetic waves sourced by a communication device external to the body.

Charging the capacitor may further include harvesting electrical energy from electrical current sourced by the communication device.

The methods of inducing hyperthermia can further include creating thermal energy by a resistive element defined on a substrate of the microchip device, and conducting the thermal energy from the microchip device to the cells proximate the microchip device.

The methods of inducing hyperthermia can further include flowing electrical current through the cells by way of set of electrodes defined on a substrate of the microchip device. Flowing the electrical may further include flowing electrical current between the set of electrodes spaced apart by 1000 microns or less. Flowing the electrical may further include flowing electrical current between the set of electrodes spaced apart by 10 microns or less.

The method of inducing hyperthermia can further include receiving a message by a communication circuit defined on the microchip device, and triggering the inducing hyperthermia responsive to the message.

The method of inducing hyperthermia can further include sensing, by the microchip device, whether the cells proximate to the microchip device are cancer cells. If the cells are cancer cells, the method may include triggering the inducing of hyperthermia.

The method of inducing hyperthermia can further include sensing a property of the cells. The property may be one or combinations of: pH; resistivity; conductivity; impedance; transmittance; dielectric constant; and oxygen level or oxygen concentration.

The method of inducing hyperthermia can further include sensing (by the first microchip device) a property of the cells proximate to the first microchip device, sending a value indicative of the property to a communication device external to the body, receiving (by a communication circuit defined on the microchip device) a message from the communication device external to the body, and triggering the inducing hyperthermia based on the message.

The method of inducing hyperthermia can further include, prior to charging the capacitor and inducing hyperthermia, implanting the microchip device to be proximate to the cells. The implanting may be by injecting the microchip device way of a needle.

Other example embodiments are medical devices for inducing hyperthermia in cancer cells. The example medical devices may include a substrate of semiconductor material, a means for harvesting energy defined on the substrate, a means for wireless communication with devices external to the substrate (the means for wireless communication defined on the substrate and electrically coupled to the means for harvesting energy), a means for sensing a property of a cells proximate to the substrate (the means for sensing electrically coupled to the means for harvesting and the means for wireless communication), and a means for inducing hyperthermia in cells proximate to the substrate (the means for inducing electrically coupled the means for harvesting and the means for wireless communication).

plementations of the medical device aspects of the invention can include one or more of the following features:

The means for harvesting may further include an energy harvesting antenna defined on the substrate (the energy harvesting antenna has an operating frequency above 1 Mega Hertz (MHz)). a rectifier defined on the substrate (the rectifier electrically coupled between the energy harvesting antenna and a capacitor) where the rectifier configured to rectify alternating current energy from the energy harvesting antenna to create rectified energy stored in the capacitor, and a power management unit defined on the substrate (the power management unit coupled to the capacitor) with the power management unit configured to produce a regulated direct current (DC) voltage from rectified energy stored on the capacitor.

The means for energy harvesting may further include a set of conductive pads electrically exposed on the substrate, a rectifier defined on the substrate and electrically coupled between the second set of conductive pads and the capacitor, a power management unit defined on the substrate and electrically coupled to the capacitor. The rectifier circuit configured to rectify alternating current energy flowing through the set of conductive pads to create rectified energy stored on the capacitor. And the power management unit configured to produce a regulated DC voltage from the rectified energy stored on the capacitor.

The means for wireless communication can further include a communication antenna defined on the substrate (the communication antenna operates at a frequency above 1 Mega Hertz (MHz)), and the means for wireless communication receives commands from an external device by way of the communication antenna.

The means for inducing hyperthermia may further include a means for creating thermal energy on the substrate, the means for creating electrically coupled to the means for harvesting, and the thermal energy created on the substrate induces hyperthermia by conduction from the substrate to the cells. In some cases the means for creating thermal energy is a resistor.

The medical device may further include a means for encapsulating and electrically isolating the substrate.

The means for inducing hyperthermia may further include a first set of electrodes on the substrate and that are electrically exposed, the first set of electrodes configured to selectively couple to the means for harvesting energy. The means for inducing may induce hyperthermia by electrical current flow through the cells by way of the first set of electrodes. The first set of electrodes may be separated by 1000 microns or less. The first set of electrodes may be separated by 10 microns or less.

The means for sensing may sense one or a combination of: pH; resistivity; conductivity; impedance; transmittance; dielectric constant; and oxygen level.

The substrate defines a length greater than a width, and the width is 500 microns or less. In some cases, the width is 200 microns or less.

### BRIEF DESCRIPTION OF THE DRAWINGS

For a detailed description of example embodiments, reference will now be made to the accompanying drawings (not necessarily to scale) in which:

FIG. 1 shows a partial cross-sectional, partial block diagram, view of a system for inducing hyperthermia in accordance with at least some embodiments;

FIG. 2 shows a perspective view of a microchip device in accordance with at least some embodiments;

FIG. 3 shows a partial schematic, partial block diagram, view of the various circuits of the substrate in accordance with at least some embodiments;

FIG. 4 shows, in block diagram form, a sensor interface circuit 324 in accordance with at least some embodiments;

FIG. 5 shows a perspective view of an implantation system in accordance with at least some embodiments;

FIG. 6 shows a perspective view of a patient and an example communication device 108 in accordance with at least some embodiments; and

FIG. 7 shows a method in accordance with at least some embodiments.

### DEFINITIONS

Various terms are used to refer to particular system components. Different companies may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection or through an indirect connection via other devices and connections.

“Electromagnetic waves” shall mean alternating electric and magnetic fields propagating through a medium.

“Hyperthermia” shall mean heating of cells causing cellular death.

“Proximate,” as it relates to proximity of a microchip device and cancer cells, shall mean that the cancer cells are within one centimeter of the microchip device.

### DETAILED DESCRIPTION

The following discussion is directed to various embodiments of the invention.

Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Various embodiments are directed to systems and related methods to induce hyperthermia in cells, such as cancer cells, within a body. More particularly, various example embodiments are directed to a microchip device that is implanted or injected into the body (e.g., by way of a needle and syringe) to be proximate to cells within the body. The microchip device harvests ambient energy and stores the energy on a capacitor formed on a substrate of the microchip device. In example cases, and after implantation, the microchip device reads electrical properties of cells proximate to the microchip device, and if the cells are determined to be cancer cells the microchip device induces hyperthermia in the cancer cells. In some cases, inducing hyperthermia involves creating heat on the substrate of the microchip device (e.g., heat created by electrical current flowing through a resistive element), and the heat then transfers by conduction to the cells proximate to the substrate. In other cases, the microchip device induces hyperthermia by flowing electrical current through the cells proximate to the microchip device. Because the hyperthermia is induced based on harvesting ambient energy, the application is periodic or pulsed. The specification now turns to an example environment in which the microchip devices may be used.

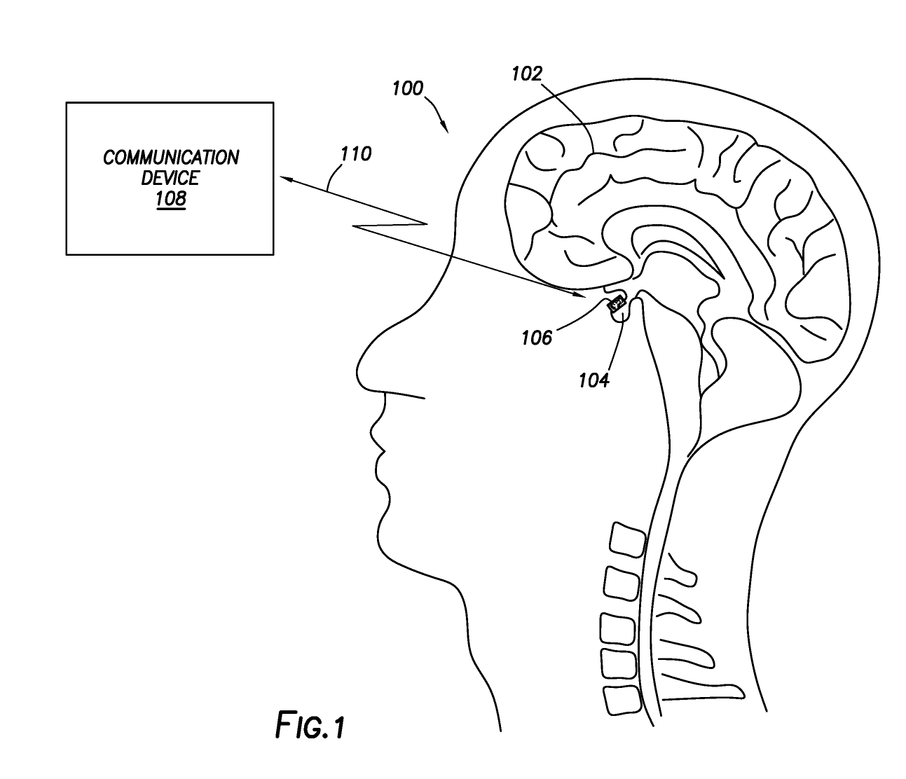

FIG. 1 shows a partial cross-sectional, partial block diagram, view of a system for inducing hyperthermia in accordance with at least some embodiments. In particular, visible in FIG. 1 is a simplified cross-sectional view of human head 100 made up of brain tissue 102 and including pituitary gland 104—the pituitary gland being one of the more common locations for the development of cancer associated with the brain. In the example system, a microchip device 106 has been placed proximate to or abutting the pituitary gland 104. FIG. 1 thus illustrates one example placement of a microchip device 106, but other placements within the skull, or in the torso of the patient, are also possible. More technically described then, in accordance with various embodiments microchip devices may be placed at any suitable location within the body where cancer cells are located, or where the cancer cells may later develop (e.g., placement after surgery to remove cancer cells, and the microchip device monitors for further cancer cell development).

In accordance with example systems the microchip device 106 (regardless of placement) does not have batteries; rather, the microchip devices in accordance with various embodiments have energy harvesting circuits that extract electrical energy from energy propagating proximate to each microchip device (hereafter “ambient energy”). The ambient energy could take many forms. For example, the communication device 108 (or other devices and systems) may send electromagnetic waves through the body that intersect the location of the microchip device 106. In other cases, the communication device 108 (or other devices and systems) may induce electrical current flow through the body that flows proximate to the microchip device 106. In yet still other cases, the communication device 108 (or other devices and systems) may launch acoustic energy toward the microchip device 106. Charging time varies based on the ambient energy available, and could be from a few minutes to an hour or more. The charging energy is sufficiently low (and the frequency selected) so as not to damage other bodily cells, tissue, and functions. Various example structures for harvesting ambient energy are discussed more below.

The example system thus further comprises communication device 108. Communication device 108 may be communicatively coupled to the microchip device 106. In particular, the communication device 108 in example embodiments is wirelessly coupled to the microchip device 106, as illustrated by double-headed arrow 110. Various mechanisms for wireless communication between the communication device 108 and the microchip device 106 are discussed more below. Suffice it to say at this stage that the communication device 108 may communicate individually to the microchip device 106 (and other microchip devices not specifically shown). The communication device 108 may take many forms. In some cases the communication device 108 resides outside the body containing the example pituitary gland 104, and is physically placed abutting or proximate to the patient's skin. In other cases, the communication device 108 may be implanted under the patient's skin, such as subcutaneous placement. In yet still other cases, the functionality of the communication device 108 may be divided between a subcutaneously placed portion and an external portion, with the two portions communicatively coupled.

In some example systems, after insertion into the body the microchip device 106 operates autonomously, sensing or detecting the presence of cancer cells, and inducing hyperthermia in the cancer cells proximate to the microchip device 106—limited only by an amount of time needed to scavenge or harvest energy for application of the next hyperthermia inducing event. Thus, in the autonomous versions, the microchip devices may omit a communication circuit, discussed more below. In other cases, the microchip device 106 operates autonomously, detecting cancer cells and inducing hyperthermia in the cancer cells proximate to the microchip device 106, but reports findings and hyperthermia inducing events to the communication device 108. In yet still other example systems, the communication device 108 controls the microchip device 106 by commanding the microchip device 106 at each step of the process. Consider, for example, the case of a microchip device 106 first commanded to sense a property of cells proximate to the microchip device 106 (sensing properties, discussed more below). The microchip device 106 may then communicate the results to the communication device 108, which makes the determination regarding the presence or absence of cancer cells proximate to the microchip device 106. If cancer cells are present, the communication device 108 may command the microchip device 106 to induce hyperthermia in the cancer cells (either immediately, or after sufficient energy has been harvested). Once the hyperthermia inducement has concluded the process may begin again.

In some example situations one or more microchip devices may be injected or otherwise implanted into a location where cancer cells are known to be present. The microchip devices may then harvest energy and induce hyperthermia in the cancer cells. Periodically, or perhaps before each hyperthermia inducing event, each microchip device could sense electrical properties of cells proximate to the microchip device, and make a determination as to whether cells proximate to the microchip device are still cancer cells (the determination made either internally or with the help of an external communication device 108), with hyperthermia inducing events ceased if no cancer cells are present. In yet still other cases, one or more microchip devices may be injected or implanted in such a way as to detect whether cancer has begun to regrow in a particular area. For example, after a surgical procedure to remove a cancerous mass, one or more microchip devices may be placed in the surrounding tissue at a location where the cancer has yet to spread. The microchip devices may periodically sense electrical properties of cells proximate to the microchip devices, and each make a determination as to whether cells proximate to the microchip device have become cancer cells (the determination made either internally or with the help of an external communication device 108). If new cancer growth is thus detected, the one or more microchip devices may inform the communication device by way of wireless communication, and also induce hyperthermia in the cells to reduce or eliminate the further cancer growth. The specification now turns to a description of an example microchip device 106.

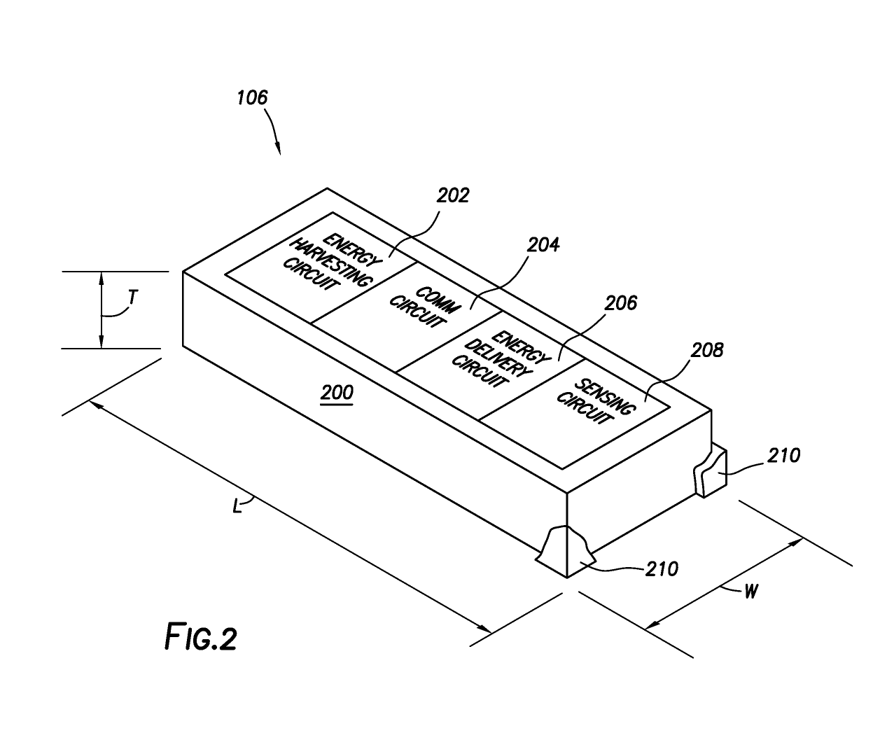

FIG. 2 shows a perspective view of a microchip device 106 in accordance with example embodiments. In particular, visible in FIG. 2 is a substrate 200 of semiconductor material, such as silicon. Constructed on the substrate, using semiconductor manufacturing techniques, are various circuits shown in block diagram form and conceptually divided into an energy harvesting circuit 202, a communication circuit 204, an energy delivery circuit 206, and a sensing circuit 208. The circuits are discussed in greater detail below. In some cases, the microchip device 106 may be encapsulated in a biocompatible material 210, though the biocompatible material 210 is shown only on two corners so as not to obscure the other components. For microchip devices that do not require electrical contact with the surrounding tissue (e.g., hyperthermia induced by conduction and energy harvesting from electromagnetic waves), the microchip device 106 may be fully encapsulated by the biocompatible material 210, which biocompatible material may be electrically insulating. In cases where at least some electrical contact with the surrounding tissue is used (e.g., electrical current based hyperthermia, and sensing of electrical properties of surrounding tissue), the microchip device 106 may be largely encapsulated by the biocompatible material 210, with windows created in the biocompatible material 210 to enable electrical contact conductive pads (discussed more below) with the surrounding tissue.

The energy harvesting circuit 202 is defined on the substrate and is electrically coupled to a capacitor (not shown in FIG. 2). The energy harvesting circuit 202 is configured to extract electrical energy from energy propagating proximate to the microchip device 106 and to store electrical energy on the capacitor of the energy harvesting circuit 202. Electrical energy stored in the capacitor thus provides operational power to the remaining circuits of the example microchip device 106. The communication circuit 204 is defined on the substrate 200 and is electrically coupled to the energy harvesting circuit 202, from which the communication circuit 204 is provided power. The communication circuit 204 is configured to receive commands originating external to the microchip device 106, such as from the communication device 108 (FIG. 1). In some cases, the communication circuit 204 may send messages to the communication device 108, such as messages indicating the electrical properties of surrounding tissue as sensed by the sensing circuit 208. The energy delivery circuit 206 is defined on the substrate 200 and is electrically coupled to the energy harvesting circuit 202, from which the energy delivery circuit 206 is provided operational power as well as electrical energy to induce hyperthermia in cells proximate to the microchip device 106. Further, the energy delivery circuit 206 is communicatively coupled to the communication circuit 204, from which the energy delivery circuit 206 may receive commands to induce hyperthermia. The sensing circuit 208 is defined on the substrate 200, is electrically coupled to the energy harvesting circuit 202, and is communicatively coupled to the communication circuit 204. The sensing circuit 208 is configured to sense electrical properties of cells proximate to or abutting the microchip device 106, and in some cases the sensing circuit is configured to trigger the communication circuit 204 to send a message responsive to the determination.

The microchip device 106 of FIG. 2 shows an example device having the ability to sense electrical properties of cells proximate to the microchip device 106, and also induce hyperthermia if the cells are determined to be cancer cells. However, in other example cases a microchip device 106 may have only sensing capability, and such a microchip device would omit the energy delivery circuit 206. Microchip devices that implement sensing only may nevertheless be deployed in conjunction with microchip devices that implement energy delivery for purposes of inducing hyperthermia. Further still, other example microchip devices may have energy delivery capability but omit the sensing circuit 208. Microchip devices 106 that implement energy delivery and not sensing may nevertheless be deployed with microchip devices that implement sensing such that the communication device 108 (FIG. 1) receives indications of electrical properties and/or presence of cancer cells, and can command other microchip devices that do not have sensing capability to induce hyperthermia. The discussion that follows assumes a microchip device with both sensing and energy delivery capability (thus implementing both the energy delivery circuit 206 and the sensing circuit 208), but such an assumption shall not be read to require both circuits in every microchip device.

The example microchip device 106 of FIG. 2 defines a length L, a width W, and a thickness T. In some example cases the microchip device 106 is designed to be implanted into the body by way of a needle and syringe-type structure, and thus the microchip device may be limited in width and thickness to fit through the needle. In some cases, needles used to inject a microchip device 106 may have an inside diameter (ID) of about 500 microns, and thus the width W of the example microchip device 106 may be slightly smaller than the ID of the needle, or about 500 microns or less. In some cases the width W may be 10 microns or less. Likewise, the thickness T of the example microchip device 106 may be slightly smaller than the ID of the needle, or about 200 microns or less, and in some cases the thickness T may be 10 microns or less. The length L is not constrained by the needle, but may be limited by fragility of the underlying substrate 200. In some cases the length L may be 10 millimeters or less, and in some cases one millimeter.

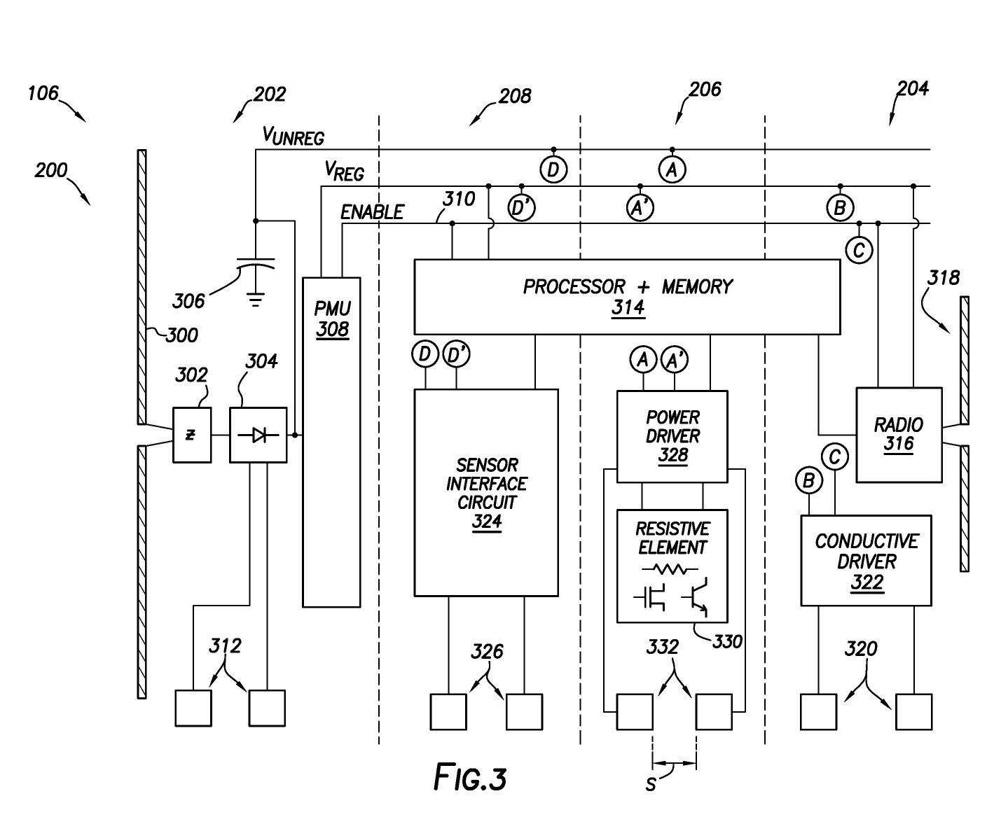

FIG. 3 shows a partial schematic, partial block diagram, view of the various circuits of the substrate in accordance with at least some embodiments. In particular, FIG. 3 shows a set of example circuits that may be monolithically constructed on a substrate of semiconductor material, such as silicon. For purposes of description, the various circuits have been conceptually, and to some extent physically, separated in the view of FIG. 3. However, some example components are shared (e.g., the processor and memory) and thus the conceptual division for purposes of description shall not be read to require physical segregation in the operable microchip device 106. With the caveats in mind, FIG. 3 shows a substrate 200 of a microchip device 106 in accordance with at least some embodiments. Shown in FIG. 3 is the energy harvesting circuit 202, the communication circuit 204, the energy delivery circuit 206, and the sensing circuit 208. Each will be discussed in turn.

Consider first the energy harvesting circuit 202. In the various embodiments the microchip device 106 harvests ambient energy to provide operational power to the other devices and components on the substrate. In some cases, the microchip device 106 harvests ambient energy in the form of electromagnetic waves propagating near, around, and/or past the microchip device 106. To that end, some example energy harvesting circuits 202 implement an energy harvesting antenna 300 illustratively shown as a dipole antenna. In example cases, the energy harvesting antenna 300 has an operating frequency of 1 Mega-Hertz (MHz) or above, in some cases having an operating frequency of between 1 MHz and 10 GigaHertz (GHz) inclusive, and in specific cases between 100 MHz and 1 GHz inclusive. The energy harvesting antenna 300 may be monolithically created on the substrate 200 by deposition of metallic material and selective etching to create metallic conductors. Other monolithically created antenna types may be equivalently used, such as bow tie antennas and patch antennas.

The energy harvesting antenna 300 electrically couples to an impedance matching network 302 (shown in block diagram form and labeled “Z”). As the name implies, the impedance matching network 302 matches impedance between the energy harvesting antenna 300 and the downstream devices to ensure low reflected energy and thus efficient energy transfer to the downstream devices. The impedance matching network 302, in turn, electrically couples to the rectifier 304. The rectifier 304 rectifies the alternating current energy from the energy harvesting antenna 300, and applies the energy to capacitor 306. The block diagram form showing the rectifier 304 illustratively shows a single diode; however, the rectifier may take any suitable form, including the half-wave rectification by way of a single diode, full-wave rectification by way of a diode bridge, and rectification by switches operated as diodes (to reduce energy loss in the form of diode voltage drop). In some cases, the rectifier 304 directly applies the rectified energy to the capacitor 306, but in other cases the rectifier 304 may further include circuitry to increase the voltage, such as a Dickson Charge Pump. In either event the rectified energy (with or without voltage step-up) is applied to the capacitor 306. The voltage on the capacitor 306 is referred to herein as the unregulated voltage (V_{UNREG}), and in some cases may be on the order of 1.6 Volts when fully charged.

The example energy harvesting circuit 202 further comprises a power management unit (PMU) 308 defined on the substrate 200. The power management unit 308 is electrically coupled to the capacitor 306, and thus is electrically coupled to the unregulated voltage. In example systems, the power management unit 308 comprises one or more circuits that selectively produce a regulated voltage (V_{REG}) from the unregulated voltage. In some cases the regulated voltage may be about 1.0 Volt. The example power management unit 308 also produces an enable signal 310 coupled to various other of the circuits. In accordance with example embodiments, the power management unit 308 de-asserts the enable signal 310 during periods of time when the energy stored on the capacitor 306 is below a predetermined threshold. With the remaining circuits disabled and thus not consuming power or consuming significantly reduced power, the energy harvesting circuit 202 can more quickly charge the capacitor 306 from ambient energy. Once the energy stored reaches or exceeds the predetermined threshold (again, e.g., 1.6 V), the power management unit 308 asserts the enable signal 310 thus enabling the remaining circuits to operate, such as sensing electrical properties by the sensing circuit 208, inducing hyperthermia by the energy delivery circuit 206, and sending and/or receiving communications by way of the communication circuit 204.

Still referring to FIG. 3, the example energy harvesting circuit 202, in addition to or in place of harvesting ambient energy in the form of electromagnetic waves, may also be designed and constructed to harvest ambient energy in the form of electrical current flowing proximate to the microchip device 106. In particular, further example systems implement a set of conductive pads 312 electrically coupled to the rectifier. Thus, when present, the conductive pads 312 are electrically exposed to the cells and tissue surrounding the microchip device 106 (such as exposed through windows in the biocompatible material 210 (FIG. 2)).

In operation, the communication device 108 (FIG. 1, alone or in combination with other devices) may create charging electrical current flow through the body and around the microchip device 108, the charging electrical current flow having a frequency in the range of 1 Hz to 10 MHz inclusive, and in some cases between 10 kilo-Hertz (kHz) and 1 MHz inclusive. Thus, the energy harvesting circuit 202 may harvest ambient energy directed through the patient for the specific purpose of charging the microchip devices. The specification now turns to the example communication circuit 204.

FIG. 3 further shows a communication circuit 204. The communication circuit 204 is defined on the substrate 200 and is electrically coupled to the energy harvesting circuit 202 (e.g., electrically coupled to the regulated voltage V_{REG}). The communication circuit comprises the processor and memory 314 (hereafter just processor 314), radio 316, and a communication antenna 318. At least a portion of the functionality of the communication circuit 204 is implemented by programs executed on the processor 314, such as formulating messages to be sent to the communication device 108, and implementing commands received from the communication device 108. Radio 316 is communicatively coupled to the processor 314, is coupled to the regulated voltage V_{REG} to receive operational power, and likewise may be coupled to the enable signal 310. The radio 316 takes packet-based messages created by the processor 314 (e.g., indications of electrical properties sensed by the sensing circuit 208) and modulates the messages for transmission. Likewise, messages received by the radio 316 (e.g., commands to induce hyperthermia) are demodulated and passed to the processor 314, which in turn implements the commands.

To send and receive messages, the radio 316 is electrically coupled to communication antenna 318, illustratively shown as a dipole antenna. In example cases, the communication antenna 318 has an operating frequency above 1 MHz, in some cases having an operating frequency of between 1 MHz and 1 Giga-Hertz (GHz) inclusive, and in specific cases between 100 MHz and 1 GHz inclusive. The communication antenna 318 may be monolithically created on the substrate 200 by deposition of metallic material and selective etching to create metallic conductors. Other monolithically created antenna types may be equivalently used, such as bow tie antennas and patch antennas.

Still referring to FIG. 3, the communication circuit 204, in addition to or in place of communication by way of electromagnetic waves, may also be designed and constructed to communicate by inducing electrical current flow in the conductive environment of the body, such that the communication device 108 can either detect the current flow directly, or the communication device may be able to detect electric fields caused by the induced electrical current flow. In particular, further example systems implement a set of conductive pads 320 electrically coupled to a conductive driver circuit 322. Thus, when present, the conductive pads 320 are electrically exposed to the cells and tissue surrounding the microchip device 106 (such as exposed through windows in the biocompatible material 210 (FIG. 2)). In operation, communicative electrical current flow by and between the communication device 108 and the communication circuit 204 may travel through the patient's body. The communicative electrical current flow may have a frequency in the range of 1 kHz to 1 MHz inclusive, and in some cases between 10 kHz and above to reduce interference with other bodily functions and systems. Thus, the conductive driver circuit 322 takes packet-based messages created by the processor 314 (e.g., indications of electrical properties sensed by the sensing circuit 208) and modulates the messages for transmission by way of electrical current flow. Likewise, messages received by the conductive driver circuit 322 (e.g., commands to induce hyperthermia) are demodulated and passed to the processor 314, which in turn implements the commands.

FIG. 3 further shows sensing circuit 208. The sensing circuit 208 is defined on the substrate 200 and is electrically coupled to the energy harvesting circuit 202 (e.g., the regulated voltage V_{REG}), and in some cases may be electrically coupled to the unregulated voltage V_{UNREG}. The sensing circuit 208 may include the processor 314, a sensor interface circuit 324 (discussed more below), and in some cases a set of conductive pads 326. Thus, when present, the conductive pads 326 are electrically exposed to the cells and tissue surrounding the microchip device 106 (such as exposed through windows in the biocompatible material 210 (FIG. 2)). Moreover, in at least some embodiments a portion of the functionality of the sensing circuit 208 may be implemented by programs executed on the processor 314, such as receiving indications of electrical properties and sending the information to the communication device 108 by way of the communication circuit 204 (in any or all the various forms).

The sensor interface circuit 324 may sense electrical properties of the cells by way of the conductive pads 326. For example, the sensor interface circuit 324 may sense localized pH (as voltage across the conductive pads 326 where one conductive pad is glass covered and sensitive to hydrogen-ion concentration, and the second conductive pad is a reference electrode). In other cases the electrical properties sensed are responsive to applying voltage and/or current to the cells by way of the conductive pads 326. For example, the sensor interface circuit 324 may apply a voltage (e.g., direct current (DC), alternating current (AC), or a voltage pulse or impulse) and then sense the electrical current response to determine electrical properties such as resistance, complex impedance, conductivity, and dielectric constant. Example circuits implemented by the sensor interface circuit 324 are discussed more below.

FIG. 3 further shows energy delivery circuit 206 defined on the substrate 200. The energy delivery circuit 206 may include the processor 314, and thus at least a portion of the functionality of the energy delivery circuit 206 is implemented by programs executed on the processor 314 (e.g., receiving instructions to induce hyperthermia). The energy delivery circuit 206 comprises a power driver circuit 328 electrically coupled to the regulated voltage V_{REG} and/or the unregulated voltage V_{UNREG}. The power driver circuit 328, under command of the processor 314, uses electrical energy to induce hyperthermia in cells proximate to the substrate 200. In some cases, the power driver circuit 328 applies electrical energy to a resistive element 330. The electrical energy applied to the resistive element 330 creates heat on the substrate, which heat then propagates to the surrounding tissue by conduction. The resistive element 330 may take any suitable form. For example, the resistive element may be a resistor constructed on the substrate, and when voltage is applied the resistor creates heat. In other cases, the resistive element may be a transistor monolithically constructed on the substrate 200, and the transistor biased into an active region. The transistor may be, for example, a bipolar junction transistor or a complementary metal-oxide semiconductor transistor. In the case of creating heat by way of the resistive element 330 (and assuming others of the various circuits do not electrically contact with the surrounding cells and tissue), the entire microchip device 106 may be encapsulated with a biocompatible material 210 (FIG. 2) that is also electrically insulating.

Inducing hyperthermia in cells is both a time- and temperature-based operation. The shorter the time of application of increased temperature, the greater the temperature needed to induce cellular death. Conversely, the longer the time of exposure to increased temperature, the lower the increased temperature needed to induce cellular death. For example, it may be possible to induce hyperthermia in cells by application of an increase over ambient body temperature of 20 degrees Fahrenheit (e.g., about 118 degrees Fahrenheit) for as short as one millisecond. Thus, in some cases creating the heat for inducing hyperthermia may involve directly coupling the resistive element 330 to the regulated voltage V_{REG} and/or the unregulated voltage V_{UNREG} until the energy stored on the capacitor 306 is depleted—creating a high temperature increase for a short period of time. Likewise, it may be possible to induce hyperthermia by application of a lower temperature increase (e.g., 10 degrees Fahrenheit) for an extended period of time, such as 10 milliseconds. Thus, in yet still other cases the power driver circuit 328 may regulate energy delivery to the resistive element 330 (e.g., pulse width modulating the applied voltage, or controlling the resistance across the resistive element in the form of a transistor by controlling the bias current and/or voltage at the gate or base)—creating a lower temperature increase but for a longer period of time.

In addition to, or in place of, creating heat by way of resistive element 330 on the substrate, the energy delivery circuit 206 may create heat in the cells proximate to the microchip device 106 by causing electrical current flow through the cells. Thus, in yet still further embodiments the energy delivery circuit 206 may comprise electrodes 332. It follows that, when present, the electrodes 332 are electrically exposed to the cells and tissue surrounding the microchip device 106 (such as exposed through windows in the biocompatible material 210 (FIG. 2)). The electrodes 332 are electrically coupled to the power driver circuit 328 such that the power driver circuit 328 may apply the regulated voltage V_{REG} and/or the unregulated voltage V_{UNREG} to the electrodes 332. Application of the voltage to the electrodes causes an electrical current flow through the cells proximate to the microchip device and thus resistive heating of the cells which induces hyperthermia. As discussed with respect to heat created by resistive element 330, inducing hyperthermia in cells is both a time- and temperature-based function. Thus, in some cases creating the heat for inducing hyperthermia may involve directly coupling the regulated voltage V_{REG} and/or the unregulated voltage V_{UNREG} to the electrodes 332 to produce as much electrical current flow as the regulated voltage V_{REG} and/or the unregulated voltage V_{UNREG} can source given the impedance of the cells proximate to the microchip device 106. In yet still other cases the power driver circuit 328 may regulate energy delivery to electrodes 332 (e.g., pulse width modulating the applied voltage) thus creating a lower temperature increase but for a longer period of time.

In some cases, the power driver circuit 328 applies the voltage from the capacitor to the electrodes 332 in a DC sense—resulting in the electrical current from the capacitor flowing from one electrode to the other without change of direction. In other cases, the power driver circuit 328 may implement a switch bridge such that the voltage is applied in an AC sense—resulting in electrical current flow first in one direction, and then the other direction, and so on. Stated slightly differently, during periods of time when electrical current flows through the tissue and cells, the power driver circuit may operate switches to alternate the polarity of the voltage that induces the electrical current flow.

The electrical current flow through the tissue and cells between the electrodes 332 is dictated, at least in part, by the voltage applied and the impedance of the underlying tissue. However, for an assumed voltage level, energy dissipated (and thus heat created) by the electrical current flow in the cells and tissue increases with decreasing impedance. Thus, in some embodiments the electrodes 332 are constructed to be relatively close together to limit the presented impedance. Stated slightly differently, assuming an impedance per unit distance of the cells proximate to the electrodes 332, closer spacing of the electrodes 332 results in lower impedance between the electrodes (and thus higher delivered power for a constant voltage). Thus, in some cases the spacing S between the closest points of the electrodes 332 may be 1000 microns or less, and in some cases 10 microns or less, and in a particular case the spacing S may be 2 microns or less (but greater than zero).

Finally with respect to FIG. 3, the various embodiments discussed to this point have assumed that the heat to induce hyperthermia is generated onboard the substrate by way of the resistive element 330, or by electrical current flow through the tissue across the electrodes. However, in yet still further embodiments inducing hyperthermia may involve both conduction of heat created by the resistive element 330 and electrical current flow through the electrodes 332. For example, the application may be simultaneous. In other cases, initial heat may be created using one method (e.g., initially electrical current flow through the tissue and cells to generate fast temperature rise), and then followed contiguously by the other (e.g., temperature maintained by conduction from the substrate). Any combination of the application of heat to induce hyperthermia may be used.

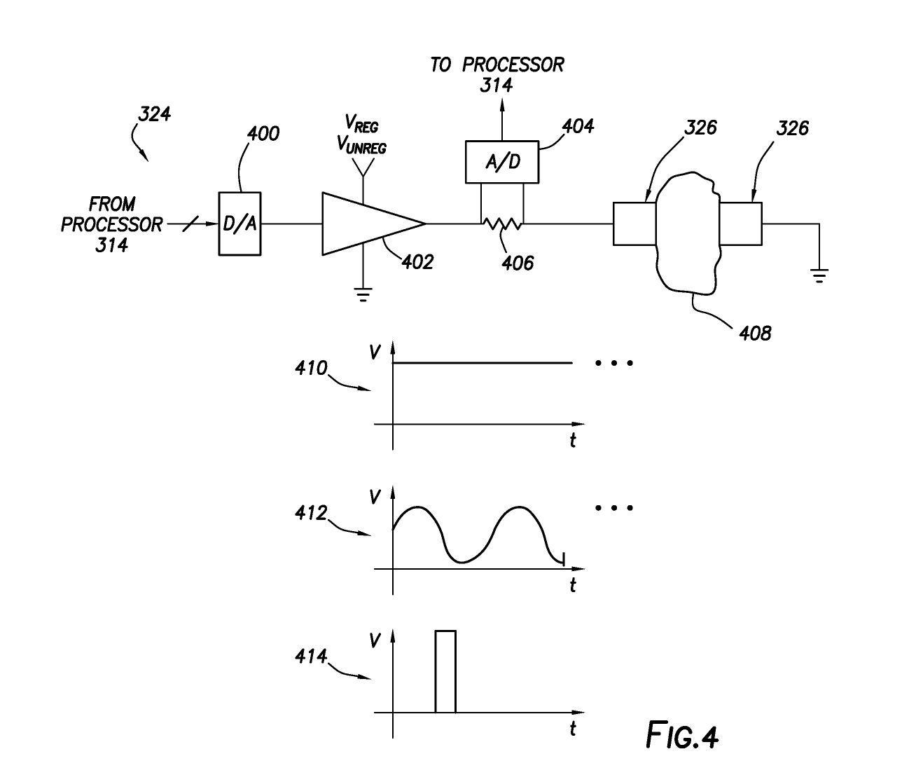

FIG. 4 shows, in block diagram form, a sensor interface circuit 324 in accordance with at least some embodiments. In particular, the example sensor interface circuit 324 comprises a digital-to-analog converter 400 that is electrically coupled to the processor 314 (FIG. 3). The analog side of the digital-to-analog converter 400 electrically couples to an amplifier 402, illustratively shown as an operational amplifier; however, any circuit that can receive an analog signal from the digital-to-analog converter 400 and increase the voltage and/or current may be used. The example amplifier 402 is coupled to an electrical current sensing circuit illustratively shown as an analog-to-digital converter 404 coupled across a small current sense resistor 406. The analog-to-digital converter 404 is electrically coupled to the processor 314 (FIG. 3) such that the processor 314 may read a voltage value indicative of electrical current through the resistor 406, and thus the electrical current flow provided by the amplifier 402. The electrical current sensing circuit electrically couples to one of the conductive pads 326, with tissue and cells 408 illustratively shown abutting each conductive pad 326. Using the example sensor interface circuit 324, the processor 314 commands the sensor interface circuit 324 to measure a property of the tissue and cells 408, such as an electrical property.

Measuring the property may take many forms. For example, in some embodiments the processor 314 may apply a DC voltage, as shown by graph 410. Thus, in these embodiments processor 314 drives the digital-to-analog converter 400 to create a constant voltage over time. The amplifier 402, deriving amplifying energy from either the regulated voltage V_{REG} and/or the unregulated voltage V_{UNREG} (e.g., having its power rails coupled to the regulated voltage V_{REG} and/or the unregulated voltage V_{UNREG} as shown), generates a DC voltage that is applied to the tissue and cells 408 by way of the conductive pads 326. Using the example analog-to-digital converter 404 and inline resistor 406, the processor 314 may thus determine the voltage applied across the conductive pads 326 (using one leg of the connection to the converter 404) and the responsive electrical current flow (as a differential voltage reading). Thus, the processor 314 may be able to calculate the electrical property of resistance (or its inverse, conductance) of the tissue and cells 408—where resistance may be indicative of whether the cells of the tissue are cancer cells.

In yet still other cases, the processor 314 may apply an AC voltage, as shown by graph 412. Thus, in these embodiments the processor 314 drives the digital-to-analog converter 400 to create a time varying voltage with a particular frequency, which frequency may be from a few kilo-Hertz (kHz) into the Mega-Hertz (MHz) range. In some cases the amplifier 402 generates a higher amplitude AC signal that is applied to the tissue and cells 408 by way of the conductive pads 326. In other cases the amplifier 402 is a voltage follower, but amplifies or increases available power to suppled the downstream devices. Using the example analog-to-digital converter 404 and inline resistance 406, the processor 314 may thus determine the voltage applied across the conductive pads 326 and the responsive electrical current flow. Thus, the processor 314 may be able to calculate the electrical property impedance (or its inverse admittance) of the tissue and cells 408—where impedance may be indicative of whether the cells of the tissue are cancer cells. Moreover, by varying the frequency of the applied voltage, the processor 314 may be able to calculate the relationship of impedance to frequency—where the relationship of impedance to frequency may be indicative of whether the cells of the tissue are cancer cells.

In yet still other cases, the processor 314 may apply voltage pulse, as shown by graph 414. Thus, in these embodiments the processor 314 drives the digital-to-analog converter 400 to create a voltage pulse. The amplifier 402 generates the signal that is applied to the tissue and cells 408 by way of the conductive pads 326. Using the example analog-to-digital converter 404 and inline resistor 406, the processor 314 may thus determine the voltage applied across the conductive pads 326 and the responsive electrical current flow during application of the voltage pulse. Moreover, the processor 314 may continue to monitor voltage across the tissue and cells 408 after the voltage pulse has ceased. Thus, the processor 314 may be able to calculate the electrical property resistance (during the pulse) as well as other electrical properties, such as dielectric strength (e.g., based on a capacitance determination and accounting for parasitic capacitance of the circuits of the microchip device). Here again, the relationship of response to the voltage pulse by the tissue and cells 408 may be indicative of whether the cells of the tissue are cancer cells.

In other cases, the property sensed may be property of the cells that is sensed electrically. As discussed above, for example, the sensing circuit 208 may be designed and constructed to sense pH. In other cases, the sensing circuit may be designed and constructed to sense oxygen level or oxygen concentration proximate to the microchip device. For example, the sensing circuit 208 of the microchip device 106 may include a Clark-type electrode, or may be designed and constructed to measure oxygen saturation using photodiodes similar to a transmission or reflectance pulse oximetry measurement. In other cases, the microchip device may implement a titanium oxide-based oxygen sensor, where the resistance of the sensor changes as a function of oxygen concentration of the tissue and cells to which the sensor is exposed. Titanium oxide-based sensors are suited to the microchip device environment because such sensors to do not require access to reference air to make the oxygen concentration measurement.

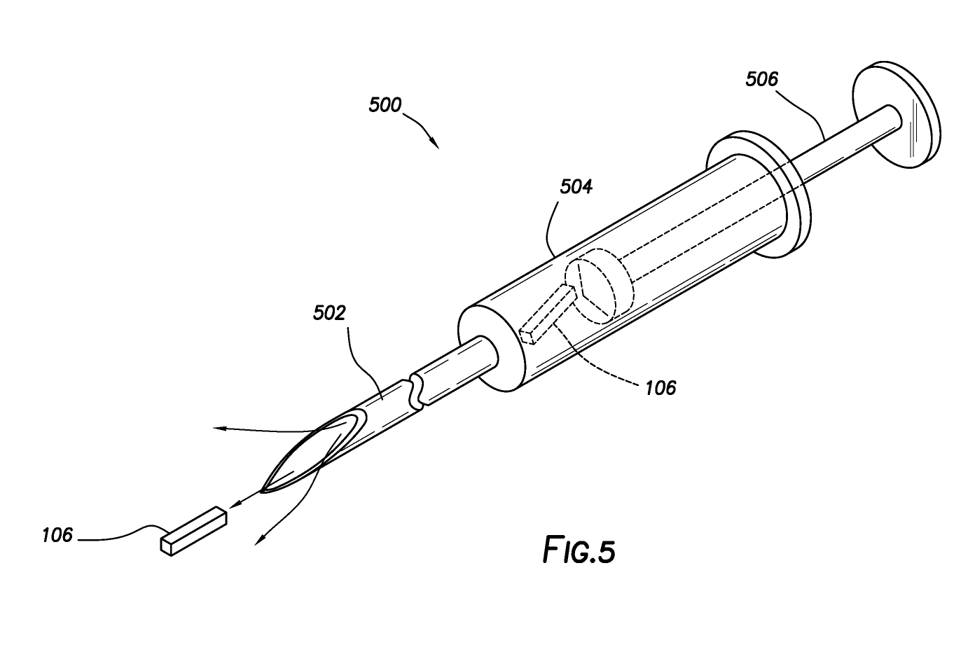

FIG. 5 shows a perspective view of an implantation system in accordance with at least some embodiments. In particular, visible in FIG. 5 is syringe system 500. The syringe system 500 comprises a needle 502 coupled to a barrel 504. The barrel 504 defines an internal volume that is fluidly coupled to the internal volume of the needle 502. A plunger 506 is operatively coupled to the internal diameter of the barrel 504 to force contents of the barrel through the needle 502. In example embodiments, the microchip devices 106 may be implanted within the body using syringe system 500. In particular, one more microchip devices 106 may be suspended in a fluid, such as saline, within the internal diameter of the barrel 504. By force applied to plunger 506, the saline and microchip device 106 may be forced through the needle 502 and into position to abut tissue within the body. FIG. 5 shows a microchip device 106 just after being ejected from the distal end of the needle 502. In yet still other cases, the microchip devices may be preloaded into the needle 502, and rather than being hydraulically forced from the needle (e.g., by using saline), each microchip device may be mechanically ejected from the needle (e.g., by a wire that pushes the microchip device(s) out of the needle). Even in the mechanical ejection and placement embodiments, the microchip devices within the needle may nevertheless be surrounded by a fluid to reduce the chances of inserting unwanted air bubbles into the patient.

In yet still other cases, the microchip devices may be placed directly. For example, in the situation where microchip devices are placed during a surgical procedure to remove cancerous tissue, the microchip devices may be physically placed by the surgeon at various locations to monitor for re-growth of the cancer cells (and possibly inducing hyperthermia when such cells are detected) without the use of the syringe system 500 noted above. Any physical system and method that places the microchip devices to abut tissue may be used.

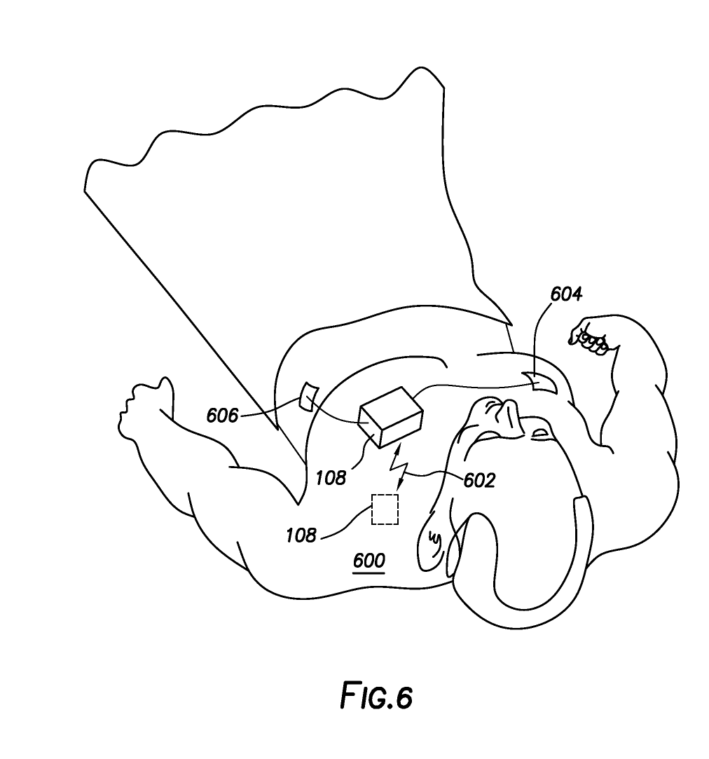

FIG. 6 shows a perspective view of a patient and an example communication device 108 in accordance with at least some embodiments. In particular, shown in FIG. 6 is a patient 600 along with several examples of a communication device 108. Rather than implantation of the microchip devices in the skull, consider in the case of FIG. 6 implantation within the chest cavity, such as microchip devices implanted to abut a tumor associated with the heart. In some cases, the communication device may be implanted subcutaneously (e.g., under the skin but outside the rib cage), as shown by communication device 108 in dashed lines. In other cases, the communication device 108 may reside fully outside the body, such as illustrated by communication device 108 shown in solid lines. In yet still other cases, the functionality of the communication device 108 may be split between a portion placed subcutaneously, and an external portion (i.e., both communication devices 108 shown in FIG. 6). In such cases, the external portion and internal portion may communicate wirelessly, as shown by arrow 602.

FIG. 6 further shows an example of communicating with the microchip devices and/or powering the microchip devices conductively. That is, FIG. 6 shows a first electrical contact 604 coupled to the chest of the patient 600, and electrically coupled to the external version of the communication device 108. A second electrical contact 606 is coupled to the rib cage of the patient 600 below the chest, thus forming a conduction path proximate to the patient's heart. By applying electrical energy across the electrical contacts (at the frequencies discussed above), the communication device 108 may power microchip devices coupled within the chest cavity. Likewise, by detecting minute voltages across the electrical contacts, the electrical fields caused by communicative electrical signals inducing current within the tissue of the patient, the microchip devices may communicate with the communication device 108. While FIG. 6 shows the electrical contacts 604 and 606, and corresponding electrical leads, external to the patient's body, in the case of the subcutaneously placed communication device 108 the leads and electrical contacts too could be subcutaneously placed. Finally, whether the communication device 108 is external, or internal, or combinations thereof, the communication device 108 may still direct electromagnetic waves to the heart to provide ambient energy for energy harvesting and/to communicate with the microchip devices.

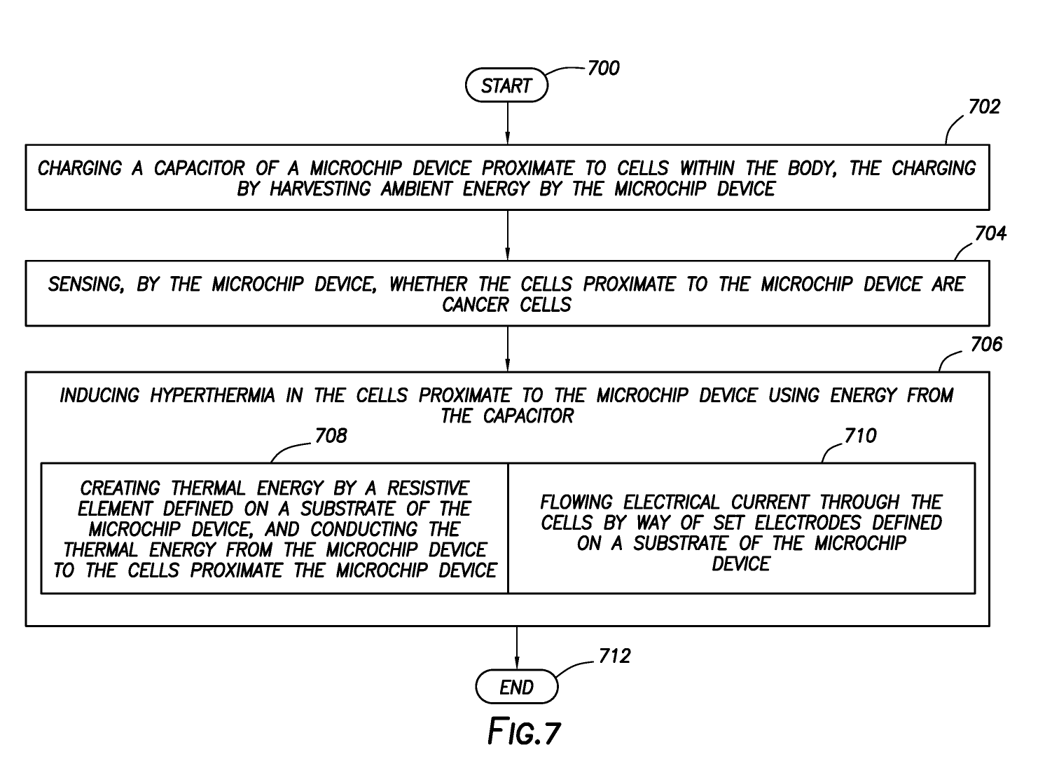

FIG. 7 shows a method in accordance with at least some embodiments. In particular, the method starts (block 700) and comprises: charging a capacitor of a microchip device proximate to cells within the body, the charging by harvesting ambient energy by the microchip device (block 702); sensing, by the microchip device, whether the cells proximate to the microchip device are cancer cells (block 704); and inducing hyperthermia in the cells proximate to the microchip device using energy from the capacitor (block 706). The inducing may take many forms, such as: creating thermal energy by a resistive element defined on a substrate of the microchip device, and conducting the thermal energy from the microchip device to the cells proximate the microchip device (block 708); and/or flowing electrical current through the cells by way of set of electrodes defined on a substrate of the microchip device (block 710). Thereafter the method ends (block 712), likely to be immediately repeated.

The above discussion regarding energy harvesting and inducing hyperthermia is meant to be illustrative of the principles and various embodiments. Numerous variations and modifications will become apparent to those skilled in the art once the above disclosure is fully appreciated. For example, the specification refers to conductive pads with respect to energy harvesting, sensing, and communications, but refers to electrodes with respect to inducing hyperthermia; however, the distinction is merely grammatical, and metallic material electrically coupled to the tissue and cells within the body may be of similar construction—such as platinum, iridium, titanium, gold, or any metallic material suitable for extended use within the body. It is intended that the following claims be interpreted to embrace all such variations and modifications.

1. An implantable medical device for inducing hyperthermia in cancer cells, the medical device comprising:
a substrate of semiconductor material;
an energy harvesting circuit defined on the substrate, the energy harvesting circuit configured to extract electrical energy from energy propagating proximate the medical device and to store electrical energy in a capacitor;
an energy delivery circuit defined on the substrate, the energy delivery circuit electrically coupled to the energy harvesting circuit, and the energy delivery circuit configured to induce hyperthermia in cells proximate to the substrate.

2. The implantable medical device of claim 1 wherein the energy delivery circuit further comprises:
a resistive element defined on the substrate and electrically coupled to the energy harvesting circuit; and
the energy delivery circuit is configured to apply heat to the tissue by conduction to the tissue of heat created from the resistive element.

3. The implantable medical device of claim 2 wherein the resistive element is at least one selected from the group comprising: a resistor; a transistor biased into an active region;
a bipolar junction transistor biased into an active region; and a complementary metal-oxide semiconductor transistor biased into an active region.

4. The implantable medical device of claim 2 further comprising an encapsulant that fully encapsulates the substrate and devices defined on the substrate, the encapsulant electrically non-conductive.

5. The implantable medical device of claim 1 wherein the energy delivery circuit further comprises:
a first set of electrodes on the substrate and electrically exposed, the first set of electrodes configured to selectively couple to the energy harvesting circuit;
the energy delivery circuit configured to induce hyperthermia by electrical current flow through the cancer cells by way of the first set of electrodes.

6. The implantable medical device of claim 5 wherein the first set of electrodes are separated by 1000 microns or less.

7. The implantable medical device of claim 5 wherein the first set of electrodes are separated by 10 microns or less.

8. The implantable medical device of claim 1 further comprising:
a communication circuit defined on the substrate, the communication circuit electrically coupled to the energy harvesting circuit and the energy delivery circuit, the communication circuit configured to receive a command originated external to the implantable medical device; and
the energy delivery circuit configured to induce hyperthermia responsive to the command received by the communication circuit.

9. The implantable medical device of claim 8 wherein the communication circuit further comprises:
a communication antenna defined on the substrate, the communication antenna operates at a frequency above 1 Mega Hertz (MHz); and
the communication circuit configured to receive the command from an external device by way of the communication antenna.

10. The implantable medical device of claim 1 wherein the energy harvesting circuit further comprises:
an energy harvesting antenna defined on the substrate, the energy harvesting antenna has an operating frequency above 1 Mega Hertz (MHz);
a rectifier defined on the substrate, the rectifier electrically coupled between the energy harvesting antenna and the capacitor, the rectifier configured to rectify alternating current energy from the energy harvesting antenna to create rectified energy stored in the capacitor; and
a power management unit defined on the substrate, the power management unit coupled to the capacitor, the power management unit configured to produce a regulated direct current (DC) voltage from rectified energy stored on the capacitor.

11. The implantable medical device of claim 1 wherein the energy harvesting circuit further comprises:
a set of conductive pads, the set of conductive pads electrically exposed on the substrate;
a rectifier defined on the substrate, the rectifier electrically coupled between the second set of conductive pads and the capacitor, the rectifier circuit configured to rectify alternating current energy flowing through the set of conductive pads to create rectified energy stored on the capacitor; and
a power management unit defined on the substrate, the power management unit coupled to the capacitor, the power management unit configured to produce a regulated DC voltage from the rectified energy stored on the capacitor.

12. The implantable medical device of claim 1 further comprising:
a sensing circuit defined on the substrate, the sensing circuit electrically coupled to the energy harvesting circuit and communicatively coupled to the communication circuit;
the sensing circuit configured to sense a property of the cells proximate to or abutting the substrate.

13. The implantable medical device of claim 12 wherein the property is at least one selected from the group comprising: pH; resistivity; conductivity; impedance; transmittance;
dielectric constant; and oxygen level.

14. The implantable medical device of claim 1 where the substrate defines a length greater than a width, and the width is 500 microns or less.

15. A method of inducing hyperthermia in cancer cells within a body, the method comprising:
charging a capacitor of a microchip device proximate to cells within the body, the charging by harvesting ambient energy by the microchip device; and when the energy on the capacitor reaches or exceeds a predetermined value
inducing hyperthermia in the cells proximate to the microchip device using energy from the capacitor.

16. The method of claim 15 wherein inducing hyperthermia further comprises:
creating thermal energy by a resistive element defined on a substrate of the microchip device; and
conducting the thermal energy from the microchip device to the cells proximate the microchip device.

17. The method of claim 15 wherein inducing hyperthermia further comprises flowing electrical current through the cells by way of a set of electrodes defined on a substrate of the microchip device.

18. The method of claim 17 wherein flowing the electrical current further comprises flowing the electrical current between the set of electrodes spaced apart by 1000 microns or less.

19. The method of claim 17 wherein flowing the electrical current further comprises flowing the electrical current between the set of electrodes spaced apart by 10 microns or less.

20. The method of claim 15 further comprising receiving a message by a communication circuit defined on the microchip device, and triggering the inducing hyperthermia responsive to the message.

21. The method of claim 15 further comprising:
sensing, by the microchip device, whether the cells proximate to the microchip device are cancer cells; and if the cells are cancer cells
triggering the inducing hyperthermia.

22. The method of claim 21 wherein sensing further comprises sensing a property of the cells.

23. The method of claim 15 further comprising:
sensing, by the first microchip device, a property of the cells proximate to the first microchip device;
sending a value indicative of the property to a communication device external to the body;
receiving, by a communication circuit defined on the microchip device, a message from the communication device external to the body; and
triggering the inducing hyperthermia based on the message.

24. The method of claim 22 wherein the property is at least one selected from the group comprising: pH; resistivity; conductivity; impedance; transmittance; dielectric constant; and oxygen level.

25. The method of claim 15 wherein charging the capacitor further comprises harvesting electrical energy from electromagnetic waves sourced by a communication device external to the body.

26. The method of claim 15 wherein charging the capacitor further comprises harvesting electrical energy from electrical current sourced by the communication device.

27. The method of claim 15 further comprising, prior to charging the capacitor and inducing hyperthermia, implanting the microchip device to be proximate to the cells.

28. The method of claim 27 wherein implanting further comprises injecting the microchip device by way of a needle.

29. A medical device for inducing hyperthermia in cancer cells, the medical device comprising:
a substrate of semiconductor material;
a means for harvesting energy defined on the substrate;
a means for wireless communication with devices external to the substrate, the means for wireless communication defined on the substrate and electrically coupled to the means for harvesting energy;
a means for sensing a property of cells proximate to the substrate, the means for sensing electrically coupled to the means for harvesting and the means for wireless communication; and
a means for inducing hyperthermia in cells proximate to the substrate, the means for inducing electrically coupled to the means for harvesting and the means for wireless communication.

30. The medical device of claim 29 wherein the means for harvesting further comprises:
an energy harvesting antenna defined on the substrate, the energy harvesting antenna has an operating frequency above 1 Mega Hertz (MHz);
a rectifier defined on the substrate, the rectifier electrically coupled between the energy harvesting antenna and a capacitor, the rectifier configured to rectify alternating current energy from the energy harvesting antenna to create rectified energy stored in the capacitor; and
a power management unit defined on the substrate, the power management unit coupled to the capacitor, the power management unit configured to produce a regulated direct current (DC) voltage from rectified energy stored on the capacitor.

31. The medical device of claim 29 wherein the means for energy harvesting further comprises:
a set of conductive pads, the set of conductive pads electrically exposed on the substrate;
a rectifier defined on the substrate, the rectifier electrically coupled between the second set of conductive pads and the capacitor, the rectifier circuit configured to rectify alternating current energy flowing through the set of conductive pads to create rectified energy stored on the capacitor; and
a power management unit defined on the substrate, the power management unit coupled to the capacitor, the power management unit configured to produce a regulated DC voltage from the rectified energy stored on the capacitor.

32. The medical device of claim 29 wherein the means for wireless communication further comprises:
a communication antenna defined on the substrate, the communication antenna operates at a frequency above 1 Mega Hertz (MHz); and
the means for wireless communication receives commands from an external device by way of the communication antenna.

33. The medical device of claim 29 wherein the means for inducing hyperthermia further comprises a means for creating thermal energy on the substrate, the means for creating electrically coupled to the means for harvesting, the thermal energy created on the substrate induces hyperthermia by conduction from the substrate to the cells.

34. The medical device of claim 33 wherein the means for creating thermal energy is a resistor.

35. The medical device of claim 33 further comprising a means for encapsulating and electrically isolating the substrate.

36. The medical device of claim 29 wherein the means for inducing hyperthermia further comprises:
a first set of electrodes on the substrate and electrically exposed, the first set of electrodes configured to selectively couple to the means for harvesting energy;
the means for inducing induces hyperthermia by electrical current flow through the cells by way of the first set of electrodes.

37. The medical device of claim 36 wherein the first set of electrodes is separated by 1000 microns or less.

38. The medical device of claim 36 wherein the first set of electrodes is separated by 10 microns or less.

39. The medical device of claim 29 wherein the means for sensing senses at least one selected from the group comprising: pH; resistivity; conductivity; impedance; transmittance; dielectric constant; and oxygen level.

40. The implantable medical device of claim 29 where the substrate defines a length greater than a width, and the width is 500 microns or less.
